(19) United States
(12) Patent Application Publication
(10) Pub. No.: US 2020/0178945 A1
(43) Pub. Date: Jun. 11, 2020

(54) Method and apparatus for minimally invasive amelioration of spinal epidural lipomatosis

(71) Applicant: Shlomo Mandol, Bloomfield Hills, MI (US)

(72) Inventor: Shlomo Mandol, Bloomfield Hills, MI (US)

(21) Appl. No.: 16/501,464

(22) Filed: Dec. 26, 2018

Related U.S. Application Data
(60) Provisional application No. 62/610,223, filed on Dec. 24, 2017.

(51) Int. Cl. A61B 10/04; A61B 17/32; A61M 1/00; A61B 17/22; A61B 1/04; A61B 1/00; A61B 1/005

(52) U.S. Cl. CPC A61B 10/04; A61B 17/320016; A61M 1/0039; A61B 2017/003; A61B 1/04; A61B 1/00119; A61B 1/0051; A61B 17/22012

(57) ABSTRACT

In an illustrative embodiment, a surgical system is provided for removing adipose buildup from epidural space of a patient spine including a surgical apparatus including a handle portion and a catheter assembly having a steerable distal tip configured for navigating epidural space of the spine; an endoscopic camera system configured to capture images from the epidural space and to identify a tissue of interest; and a vacuum source in communication with the catheter assembly of the surgical apparatus and configured to provide a negative pressure through a lumen of the catheter assembly to the tissue of interest; a discharge tank configured to capture suctioned tissue from the catheter assembly; and where the surgical apparatus further includes at least one fiber optic cable in communication with the endoscopic camera system.

### RELATED APPLICATIONS

This application claims priority to U.S. Provisional Patent Application Ser. No. 62/610,223, entitled “Method and Apparatus for Minimally Invasive Amelioration of Spinal Epidural Lipomatosis,” filed Dec. 24, 2017.

### BACKGROUND

Statistically, 9 of 10 people will experience back pain in their lifetime. While the symptoms may be similar, lower back pain actually represents a wide variety underlying causes. Unique to the skeletal system, the spine provides both structural support and protection for the central nervous system. Specifically, a boney canal surrounds the cord, extending from the cranium to the coccyx. This conduit is made up of adjacent boney vertebrae. They are in turn connected ventrally by the posterior longitudinal ligament and posteriorly by the ligamentum flavum. Hypertrophy of the ligaments can encroach the osseous canal leading to crowding of the neural elements. When arthritic inflammation develops, the swollen joint indents the dural sac and thus contributes to further narrowing of the spinal canal. Other causes of decreased canal diameter include posterior intervertebral disc displacement and space occupying cysts. The degenerative process, leading to loss of structural integrity, is well described and documented.

Circumferential canal narrowing produces congestion, known today as spinal stenosis. Epidemiologically, this is one of the most common causes of back pain and limited functional capacity. Although the physiologic basis for symptomatic stenosis is not entirely clear, decompression of the spinal canal results in effective treatment. Frequently this is accomplished by open surgery to remove anatomic structures which compress or displace the spinal cord and nerve roots. For instance, discectomy to excise extruded or bulging disc tissue. With increasing use of spine MR imaging, another important cause of spinal stenosis was identified. First described less than 40 years ago, accumulation of adipose tissue in the epidural space is currently noted by radiologists more frequently than ever before.

Spinal Epidural Lipomatosis (SEL) is a disorder characterized by increasing amount of fat tissue present within the epidural space which in turn reduces the spinal canal caliber. Pressure sensitive neural structures malfunction when forcefully pushed. SEL may lead to symptomatology described as compressive myelopathy or radiculopathy. The neurological signs produced by SEL correspond to the location of accumulated adipose tissue along the spine. Thoracic and lumbar segments are most commonly affected by fat deposition in the epidural space. Hypertrophy of epidural adipose tissue is associated with long-term steroid use. In addition to fat, important arteries and veins reside within the epidural space. Navigation in this space therefore must be cautious and visually guided. While techniques for removal of other neural compressive structures, like disc and ligamenta flava are currently in use, there is no endoscopic, percutaneous solution for SEL. Recent research suggests that unusual accumulation of fat in the epidural space has been traditionally underdiagnosed by radiologists. Lipomatosis therefore is responsible for more cases of symptomatic spinal stenosis then that is currently appreciated. Thus the need in the art for a percutaneous minimally invasive solution to removing adipose buildup from the epidural space.

### SUMMARY OF ILLUSTRATIVE EMBODIMENTS

The forgoing general description of the illustrative implementations and the following detailed description thereof are merely exemplary aspects of the teachings of this disclosure, and are not restrictive.

The present disclosure relates to surgical intervention in the epidural space (ES). More specifically the present disclosure relates to surgical intervention to correct Spinal Epidural Lipomatosis (SEL). Most specifically the present disclosure relates to minimally invasive, percutaneous surgical intervention to ameliorate the effects of SEL using liposuction.

In an exemplary embodiment, the surgical system includes a surgical apparatus having a catheter assembly configured to navigate within epidural space of the spine; an endoscopic camera system configured to illuminate and capture images from the epidural space to identify tissue of interest; and a vacuum source in communication with the catheter assembly of the surgical apparatus for suctioning the tissue of interest.

In an exemplary embodiment, the surgical system includes a surgical apparatus for removing tissue from epidural space of the spine including a catheter assembly having a steerable distal tip configured for navigating epidural space of the spine; at least one fiber optic cable in communication with an endoscopic camera system configured to allow visualization of the interior of the epidural space and identify a tissue of interest; and a vacuum source in communication with a first lumen of the catheter assembly configured to provide suction to the tissue of interest.

In an exemplary embodiment, the surgical system includes a surgical morcellator configured to break up the tissue of interest during suction.

In an exemplary embodiment, the catheter assembly is configured to be connected to the vacuum source by a semi-rigid transparent tube, where the tissue of interest can be visualized as it is suctioned.

In an exemplary embodiment, the distal tip of the catheter assembly has a blunt shape configured to prevent piercing of the thecal sac.

In an exemplary embodiment, the surgical system includes an agent source in communication with a second lumen of the catheter assembly, wherein the agent source is configured to condition the tissue of interest.

In an exemplary embodiment, a property of the tissue of interest is sensed between each intermittent suction of the tissue of interest.

In an exemplary embodiment, tissue sensing allows for confirmation of tissue type before each intermittent suction of the tissue of interest.

In an exemplary embodiment, the distal end of the canula includes an adjustable suction tip configured to modify the suction area.

In an exemplary embodiment, the surgical system includes a second suction nozzle configured to manipulate a first tissue and a second suction nozzle to resect the tissue of interest.

In an exemplary embodiment, the surgical system includes an actuator configured to vibrate the catheter assembly.

In an exemplary embodiment, the surgical system includes a marker for confirming catheter assembly within in the epidural space for imaging guidance.

In an exemplary embodiment, the vacuum source includes a pressure regulator configured to automatically stop based on a tissue type.

In an exemplary embodiment, the camera system is configured to capture two or more different wavelengths of light.

In an exemplary embodiment, the camera system includes a projection of tissue type on a display viewable by a surgeon.

In an exemplary embodiment, the surgical system includes one or more sensors configured to sense a tissue property.

In an exemplary embodiment, the one or more sensors can include a biopsy probe configured to sample tissue.

In an exemplary embodiment, the one or more sensors can include one or more electrodes configured for performing electrical recordings.

In an exemplary embodiment, each suction tip can have one or more electrodes that can interact.

In an exemplary embodiment, the one or more sensors can include one or more pressure sensors configured to detect a mechano-tissue response for identifying a tissue property.

In an exemplary embodiment, a method for removing adipose buildup from epidural space of a patient, includes locating a tissue of interest within the epidural space; guiding a catheter assembly for a surgical apparatus within the epidural space; confirming the catheter assembly entry proximal to the tissue of interest into the epidural space; and controlled suctioning of the tissue of interest.

In an exemplary embodiment, the method for removing adipose buildup from the epidural space includes at least one of an ultrasound-assisted lipectomy and radiofrequency-assisted lipectomy.

In an exemplary embodiment, the controlled suctioning is an intermittent pulsed suction.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a drawing of a build up of fat in the epidural space of a spine of a patient according to an example;

FIGS. 2A-2C are illustrations of a surgical system including a surgical apparatus in communication with a camera system, a vacuum source and a discharge tank according to an example;

FIGS. 3A-3B are illustrations of a handheld surgical apparatus including a catheter assembly according to an example;

FIG. 3C is an illustration of a distal end of the catheter assembly actuated with a number of linear actuators positioned along a length of the catheter assembly according to an example;

FIG. 3D is an illustration of a distal end of the catheter assembly actuated with a number of extenders and a number of cylindrical actuators positioned in series according to an example;

FIG. 4A is a drawing of a surgical apparatus including a camera system having a fiber that is coupled to a catheter assembly of the surgical apparatus including a lumen according to an example;

FIG. 4B is a drawing of a surgical apparatus including a camera system having a fiber that is integrated into a catheter assembly of the surgical apparatus including a lumen according to an example;

FIG. 4C is a drawing of a surgical apparatus including a camera system having a fiber that is housed within a lumen of a catheter assembly of the surgical apparatus according to an example;

FIG. 5A is a drawing of a distal end of a catheter assembly is shown having a distal face and exposing a lumen of the catheter assembly according to an example;

FIG. 5B is a drawing of a distal end of a catheter assembly is shown having a distal face and one or more sensors according to an example;

FIGS. 5C-5E are drawings of a distal end of a catheter assembly having a suction tip having one or more flaps according to an example;

FIGS. 5F-5G are drawings of a distal end of a catheter assembly having a lateral suction opening and having a shape to aid in insertion and navigation of the tissue according to an example;

FIG. 6 shows a distal end of a catheter assembly having a number of flaps controllably moved with a respective actuator and configured to conform to a surface of the tissue according to an example;

FIG. 7 is an illustration of a catheter assembly configured for linear movements towards the tissue according to an example; and

FIG. 8 is a flowchart of a method for removing adipose buildup from epidural space of a patient according to an example.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

A review of SEL's causes and recommendations for treatment are described in “Spinal epidural lipomatosis: a review of its causes and recommendations for treatment”, Fassett, et al., Neurosurg Focus 16 (4). Article 11, 2004, the contents thereof hereby incorporated by reference.

A surgical system and method are provided for removing tissue from the spinal canal. In an aspect, the surgical system is configured to provide a percutaneous surgical intervention to ameliorate the effects of Spinal Epidural Lipomatosis. The disclosed surgical system and method are provided for treating patients receiving long-term exogenous steroid therapy, patients with endogenous steroid overproduction, obesity, and idiopathic disease. In an aspect, the surgical system can be used for treating patients with other conditions resulting in hypertrophy of the epidural adipose tissue, causing a narrowing of the spinal canal and compression of neural structures.

In an exemplary embodiment, the surgical system includes a surgical apparatus having a thin and flexible liposuction catheter configured to be used endoscopically with microscopic cameras. In an example, the surgical intervention includes focused liposuction or liposculpturing. In an aspect, liposculpturing is a precise forming of tissue areas and contours. Generically, liposuction is the surgical procedure of removing fat cells or adipocytes with specially designed suction devices. Using stiff hollow catheters, the subcutaneous fat can be removed through tiny incisions. For providing percutaneous surgical intervention to ameliorate the effects of Spinal Epidural Lipomatosis, an advanced understanding of the anatomy of epidural space and adjacent structures are also essential for positive and successful clinical outcomes.

Importantly, fat in the epidural space surrounds dura mater and protects the neural structures, and facilities the movement of the dural sac. Therefore, the disclosed system and methods are used to ameliorate the symptoms of stenosis caused by buildup of fat in the epidural space, while also maintaining a healthy amount of fat. In some implementations, the determination of a healthy amount of fat is pre-determined by average thicknesses between patients (e.g., between 6-10 mm), average thicknesses at different portions of the patents' own body as determined by imaging.

In an exemplary embodiment, the progress of the treatment can be determined by intermittent imaging, checking symptoms (e.g., pain) and gathering patient feedback on their symptoms, CSF pressure above and below the surgical site, nerve conduction tests of the nerve roots.

The present disclosure combines the techniques of percutaneous spinal surgery within the epidural space using instruments that are capable of safe endoscopic navigation through the epidural space and the techniques and instruments used for liposuction. In some implementations, removal of excessive fat from the epidural space can be done in conjunction with other therapeutic measures to reduce the pressure on the spinal cord and give claudication relief.

Importantly, the disclosed methods for epidural fat removal are non-cosmetic application of fat tissue suctioning. In addition, the lipectomy procedure uniquely removes fat tissue which is attached to bone as opposed to skin or soft tissue. Direct or endoscopic visualization allows for distinguishing the spinal nerve roots from the fat tissue in the epidural space.

In an aspect, the present surgical methods and surgical apparatus can use surgical techniques such as Epiduroscopy. Epiduroscopy is fully described in chapter 7 of a book called “Pain Management”, published by Intech, available at http://www.intechopen.com/books/pain-management. The chapter is entitled “Epiduroscopy (Epidural Endoscopy)”, by Sayhan, et al., pages 115-141 the disclosure of which is hereby incorporated by reference. Epiduroscopy is also described in U.S. Pat. Nos. 6,464,682; 6,010,493; and 5,857,996, all entitled “Method of Epidural Surgery”, the contents of each being hereby incorporated by reference. While these patents describe one technique for gaining access and confirming entry of the epidural space, there are many other techniques. The journal article entitled “Localization of epidural space: A review of available technologies”, Elsharkawy, et al., J Anaesthesiol Clin Pharmacol. 2017 Jan-Mar; 33(1): 16-27, the contents thereof hereby incorporated by reference describes other techniques that can be used in the method and apparatus of the present invention.

### Surgical System

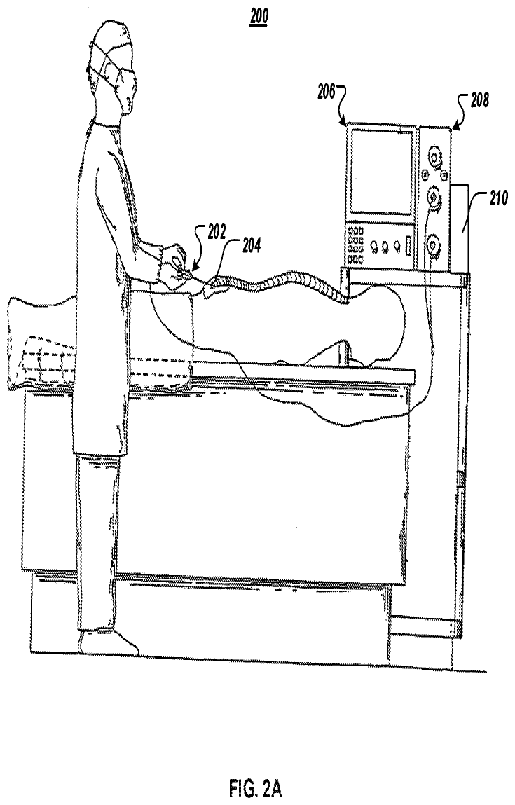

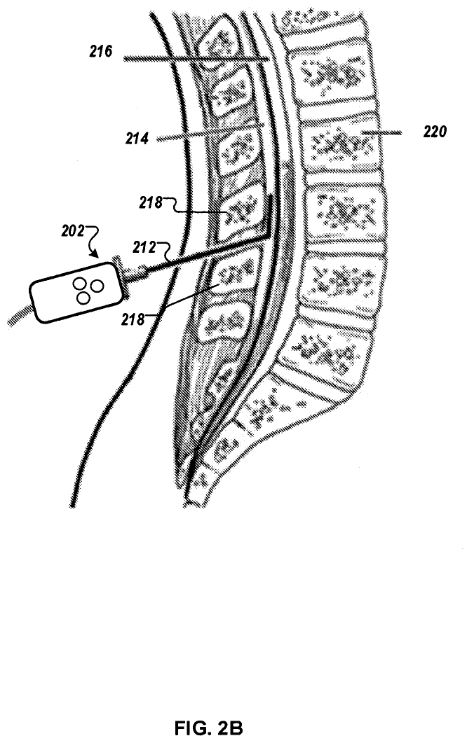

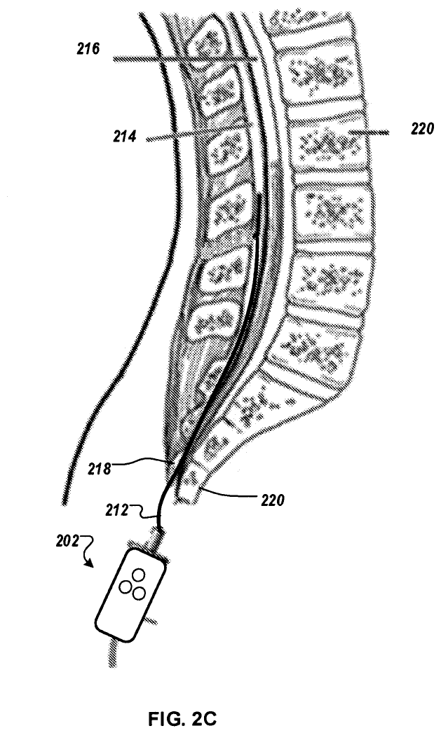

According to an exemplary embodiment, FIGS. 2A-2C illustrate a minimally invasive percutaneous spinal canal suction system or surgical system 200 includes a surgical apparatus 202 having a catheter assembly configured to navigate within epidural space 204 of the spine, an endoscopic camera system 206 configured to illuminate and capture images from the epidural space to identify target tissue, and a vacuum source 208 in communication with the catheter assembly of the surgical apparatus for suctioning the target tissue. In an example, the vacuum source 208 is in communication with a removable discharge tank 210 configured to capture suctioned tissue. FIG. 2B shows use of the system includes placement of the catheter assembly of the surgical apparatus through the mid back of a patient and into the epidural space 214 around the dura. FIG. 2C shows use of the system includes placement of the catheter assembly of the surgical apparatus through the sacral hiatus of a patient and into the epidural space 214 around the dura. As shown in FIGS. 2B-2C, through either entry, the catheter assembly 212 of the surgical apparatus 202 can be navigated and placed within the epidural space 214. Other identifying structures of the spinal column are shown including the spinal cord 216, bone 218, and vertebrae 220. Further details of the system and methods are described below.

According to an exemplary embodiment, a surgical apparatus for removing tissue from epidural space of the spine is provided including a catheter assembly having a steerable distal tip configured for navigating epidural space of the spine, at least one fiber optic cable in communication with an endoscopic camera system configured to allow visualization of the interior of the epidural space and identify a tissue of interest, and a lumen of the catheter assembly in communication with the vacuum source configured to provide suction to the tissue of interest.

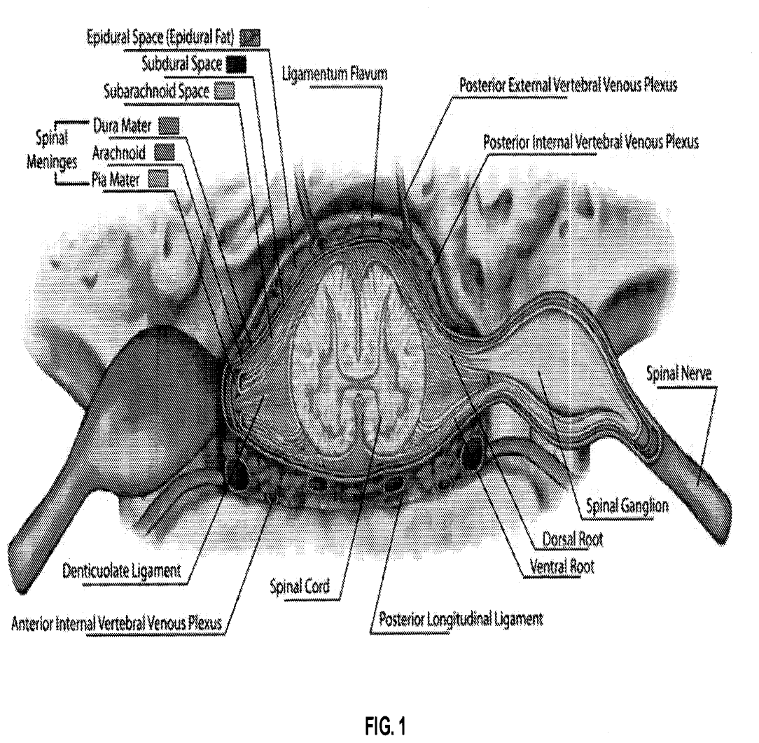

In an example, the catheter assembly can be inserted through the sacral hiatus into the epidural space around the dura and guided up toward the tissue of interest or the affected site. In an example, the catheter assembly is configured to navigate through bone landmarks and around vascular and nerve structures as shown in FIGS. 1, 2B, 2C.

In an exemplary embodiment, the surgical apparatus further includes at least one of an ultrasound-assisted lipectomy and power/radiofrequency-assisted lipectomy. Other examples of tissue ablation techniques include water-based pressure, and laser-based emulsification.

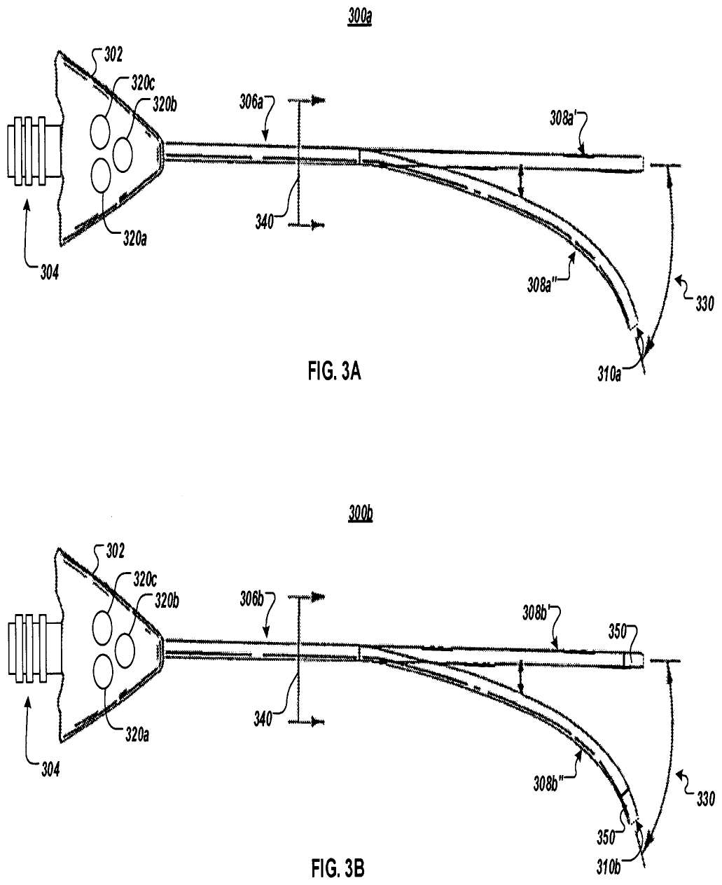

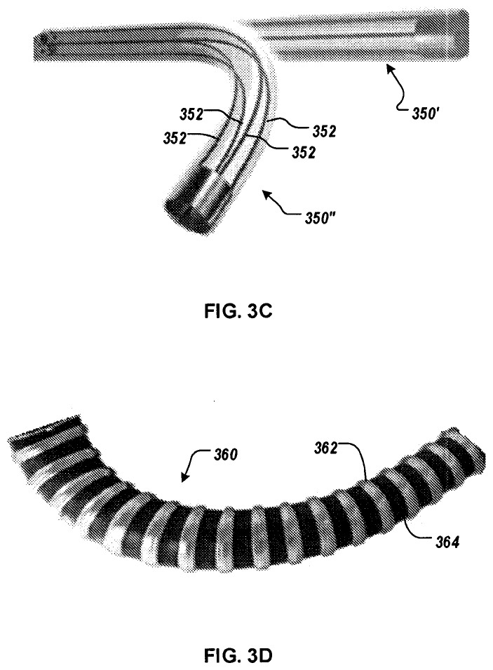

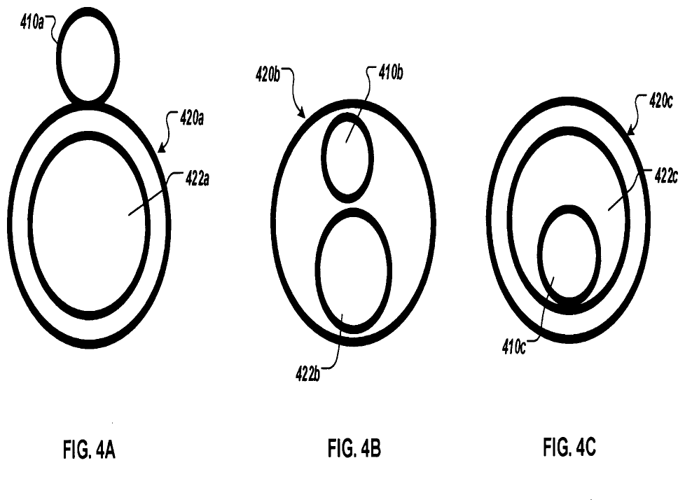

Turning to FIGS. 3A-3B, examples of a handheld surgical apparatus 300a-b are shown. In an example shown in FIG. 3A, a handheld surgical apparatus 300a includes a handle portion 302, a connector 304, and a catheter assembly 306a having a distal portion 308a. In an example, the surgical apparatus can be considered an epidural suction scope configured to remove adipose tissue safely and effectively. In an example, the handle portion 302 includes controls 320a-c configured to control at least one of suctioning, monitoring, actuating, heating, camera functions, and fine movement. A cross section of the catheter assembly 306a-b is shown at 340 and FIGS. 4A-4C.

According to an exemplary embodiment, the distal portion 308 is selectively flexible. In some implementations, the distal portion 308 further includes a suction tip 350. In some implementations, the suction tip 350 can be static and/or an adjustable suction tip configured to modify (e.g., narrow and/or widen) a suction area. In some implementations, the adjustable suction tip can be narrowed and/or widened using a shape memory material such as nitinol.

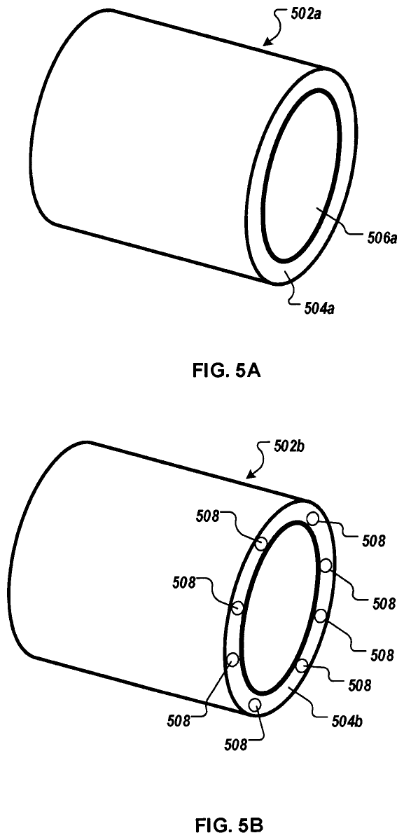

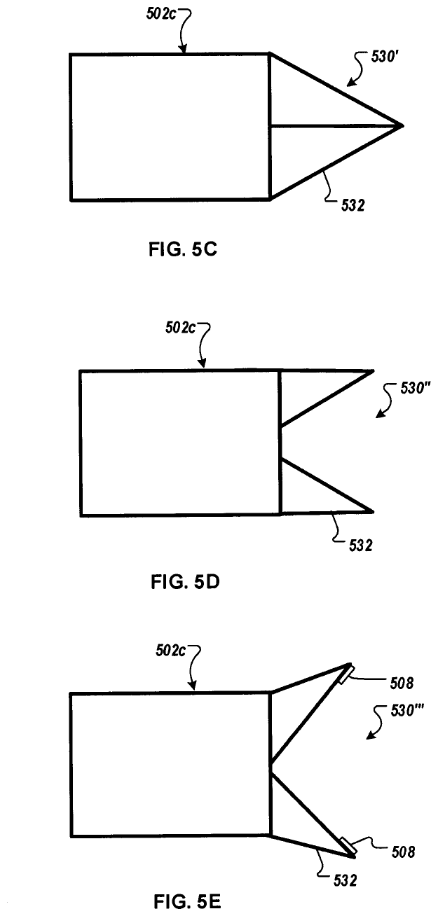

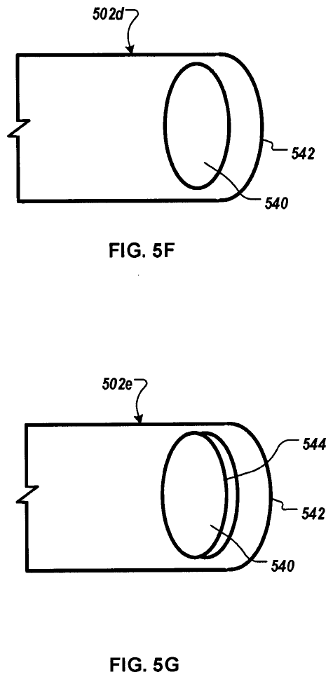

(See also FIGS. 5C-5D) In some implementations, the suction tip 350 can be molded integrally with the catheter assembly.

In an aspect, the connector 304 can be configured to connect one or more pneumatic tubes in communication with the vacuum source 208, one or more electrical elements, and one or more mechanical structures. Examples of mechanical structures include springs, gears, and other mechanical transducers communicating from the surgical system 200 to at least one of the handle portion 302, the catheter assembly 306a-b, and the suction tip 350. In an example, the one or more electrical elements can be in communication with electrical and/or electro-chemical monitoring and/or stimulation equipment.

In an example, the catheter assembly can be made from a single piece of durable plastic, metal materials, composite materials or any other suitable material. Metal materials include stainless steel, titanium alloy, duralumin, aluminum, a resin material such as a nylon resin, ABS resin, polycarbonate resin, and saturated propylene. A composite material of a metal material and a resin material may also be used. In another example, the catheter assembly can be made from multiple assembled pieces and materials.

As shown in FIGS. 3A-3B, the distal end of the catheter assembly is flexible and can be moved, guided, navigated or steered (330). In some implementations, the catheter assembly can be steered using an actuator and/or a shape memory material such as nitinol. In an example, the actuator can be positioned along a length of the catheter assembly. In some implementations, the actuator can be a separate piece attached to the catheter assembly. In some implementations, the actuator can be integrated into the catheter assembly. In some implementations, the distal end can have a ferromagnetic material which can be steered or directed using one or more magnets.

As shown in FIGS. 3C-3D, the distal end of the catheter assembly can be actuated in several ways. FIG. 3C shows a distal end of the catheter assembly 350′ actuated with a number of linear actuators positioned along a length of the catheter assembly according to an example. FIG. 3D shows a distal end of the catheter assembly 360 actuated with a number of extenders 362 and a number of cylindrical actuators 364 positioned in series according to an example. In an aspect, the alternating extenders 362 and cylindrical actuators 364 are configured to act as a snake-like stomach, applying pressure and pumping the tissue through the lumen and length of the catheter assembly.

### Camera System

According to an exemplary embodiment, the surgical system includes an endoscopic camera system configured for providing the physician or other medical personnel a detailed picture and imaging of structures within the epidural space. In an example, the endoscopic camera system includes a fiber optic scope or fiberscope. In some implementations, a surgical apparatus 400a can include a camera system having a fiber 410a that is coupled to a catheter assembly 420a of the surgical apparatus including a lumen 422a (See FIG. 4A). In this case, one or more portions of the camera system and/or the surgical apparatus can be interchanged and replaced between use. In some implementations, a surgical apparatus 400b can include a camera system having a fiber 410b that is integrated into a catheter assembly 420b of the surgical apparatus including a lumen 422b (See FIG. 4B). In some implementations, a surgical apparatus 400c can include a camera system having a fiber 410c that is housed within a lumen 422c of a catheter assembly 420c of the surgical apparatus (See FIG. 4C). In an example, two or more cameras may be placed in the epidural space for expanding the field of view and providing binocular vision.

In an example, a catheter assembly can include a light source working in conjunction with the camera system. In an example, the camera system is configured to capture two or more different wavelengths of light that can aid in identifying the tissue of interest from other tissues. In an example, the camera system includes a projection of tissue type on a display viewable by the user. vacuum source

In some implementations, the vacuum source can be any device configured to create a negative pressure. In an example, the vacuum source can be a mechanical pump configured to be controlled by a user. In an example, the vacuum source can be a syringe manually operated. In some implementations, the vacuum source can include a pressure regulator configured to automatically stop based on a tissue type. In an example, the vacuum source can be configured to generate a negative pressure to safely aspirate fat. Examples of negative pressure are substantially around −730 mmHg or greater. Based on the cross sectional areas of the tissue and lumen diameter the negative pressure can be modified accordingly. catheter assembly

In an exemplary embodiment, the surgical apparatus includes a flexible catheter assembly designed to both navigate within and suction out the fat of the epidural space. In an example, the catheter assembly can remove excess fat from around the dural sac. Importantly, the catheter assembly is preferably sized similarly to existing epidural scopes having micro-cannulas with inner diameters (ID) of 1.2 to 2.2 mm. However, portions of the catheter assembly may also be sized similar to macro-cannulas (ID 4 to 8 mm) as well as any size in-between.

In some implementations, the catheter assembly is configured to be connected to the vacuum source by a semi-rigid transparent tube, where the tissue of interest (e.g., isolated fat) can be visualized as it is suctioned.

In some implementations, the catheter assembly includes a distal tip having a blunt shape configured to prevent piercing of the thecal sac. In an example, the distal tip can be used to break up adipose tissue and facilitate suctioning.

In some implementations, the distal end of the canula includes an adjustable suction tip configured to modify the suction area.

In an example, the catheter assembly can have a blunt end with a round tip and a lateral opening to direct away from the spinal column. In an example, the catheter assembly can have a notched area for orienting positioning of the lateral opening.

Turning to FIG. 5A, a distal end 502a of a catheter assembly is shown having a distal face 504a and exposing a lumen 506a of the catheter assembly. As shown in FIG. 5B, a distal end 502b of a catheter assembly is shown having a distal face 504b and one or more sensors 508. In an aspect, the one or more sensors 508 are configured to sense a tissue property. In an example, the one or more sensors can include a biopsy probe configured to sample tissue. In an example, the one or more sensors can include one or more electrodes configured for performing electrical recordings and/or stimulation. In an example, the one or more sensors can include one or more actuators and pressure sensors configured to detect a mechano-tissue response for identifying and monitoring a tissue property. For example, an actuator can be used to perturb the tissue in isotonic and/or isometric manners. In an aspect, the tissue property includes at least one of elasticity, dimpling, or any other tissue property.

Turning to FIGS. 5C-5E, a distal end 502c of a catheter assembly is shown having a suction tip 530 having one or more flaps 532. In an example, the one or more flaps 532 of the suction tip 530 can be configured to form a shape (530′) to aid in insertion and navigation of the tissue (FIG. 5C). In an example, the one or more flaps 532 of the suction tip 530 can be configured to form a shape (530″) to aid in suction and removal of the tissue (FIG. 5D). In an example, the one or more flaps 532 of the suction tip 530 can be configured to form a shape (530″′) to aid in forming a seal with the tissue (FIG. 5E). In some implementations, each flap 532 can have one or more sensors 508. In an example, each flap on the suction tip can have one or more sensors or electrodes that can interact with another sensor or electrode on another flap.

Turning to FIGS. 5F-5G, a distal end 502d-e of a catheter assembly is shown having a lateral suction opening 540 and having a shape 542 to aid in insertion and navigation of the tissue. As shown in FIG. 5G, in some implementations, the lateral suction opening 540 can have a cutting edge 544 configured to cut tissue.

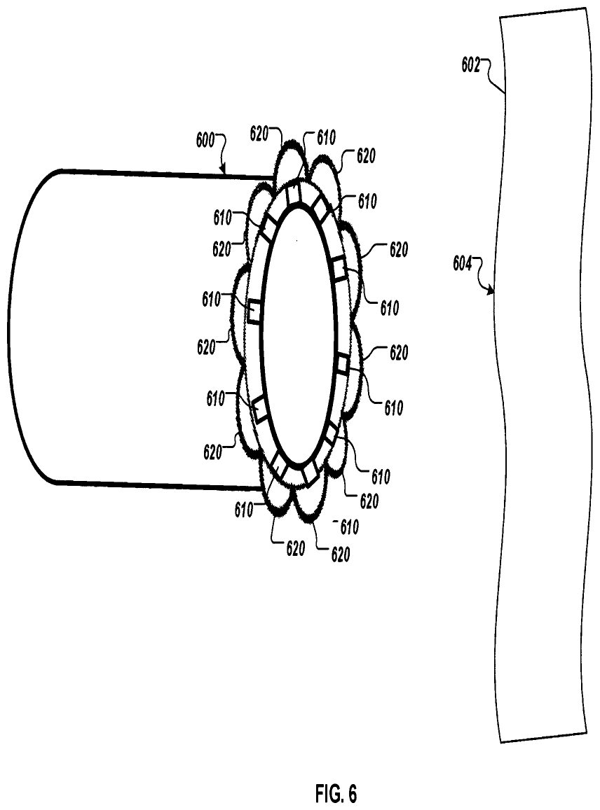

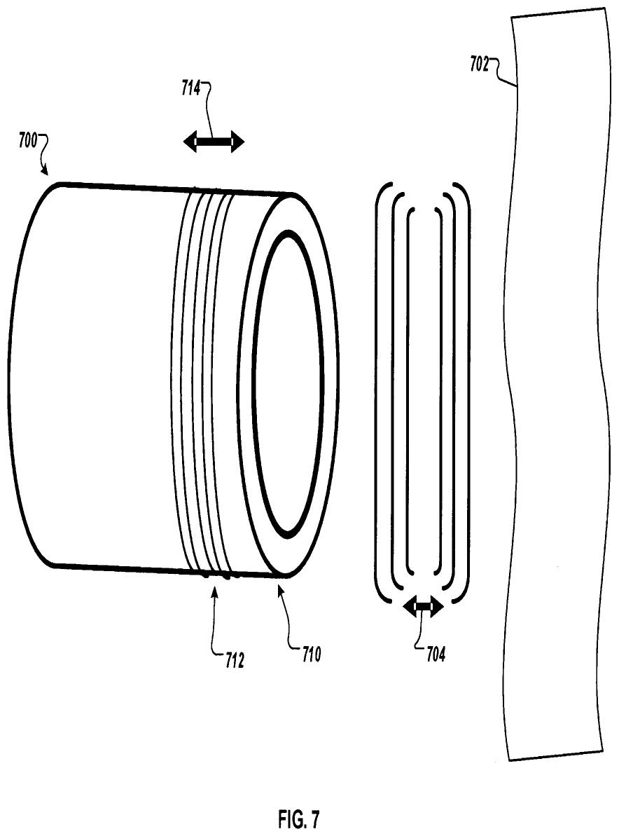

FIG. 6 shows a distal end of a catheter assembly 600 having a number of flaps 620 controllably moved with a respective actuator 610 and configured to conform to a surface 604 of the tissue 602 according to an example. In an aspect, the flaps 620 are configured to form a substantial seal with the surface 604 of the tissue 602 such that surrounding fluid is prevented from being suctioned.

In an exemplary embodiment, the surgical apparatus can include features for breaking down the suctioned tissue. In some implementations, the surgical apparatus includes a surgical morcellator (not shown) configured to break up the tissue during suctioning either near the distal tip or further down the catheter assembly and even within the handle portion 302. In an example, the suctioning can be configured for simultaneous aspiration of coagulated and liquefied fat.

In some implementations, the surgical apparatus further including an agent source in communication with a second lumen of the catheter, where the agent source is configured to condition the tissue of interest. Examples of conditioning includes heating, cooling, firming (injecting with asteroid locally to prevent swelling) as well as other techniques for isolating the tissue of interest and facilitating breakdown and removal. In an example, a laser, RF energy, and ultrasound energy can be used to heat and break up tissue.

In some implementations, the surgical apparatus can include a marker for confirming catheter assembly within in the epidural space for imaging guidance.

Turning to FIG. 7, a catheter assembly 700 is shown having a distal end 710 including a moving portion 712 configured for linear movements 714 (e.g., ultrasonic) towards the tissue 702 according to an example. In an example, the movement 714 can cause constructive and destructive waves 704 configured to augment the tissue selection and break down processes. Methods

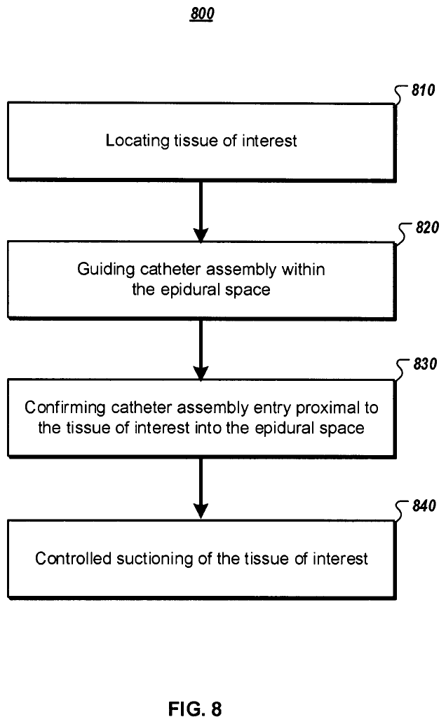

Turning to methods of use, FIG. 8 is a flowchart of a method 800 for removing adipose buildup from epidural space of a patient, the method including locating a tissue of interest (810), guiding catheter assembly within the epidural space (820), confirming catheter assembly entry proximal to the tissue of interest into the epidural space (830), and controlled suctioning of the tissue of interest (840).

In an aspect, the methods of use includes avoiding trauma to dural sac and epidural vessels. In an example, the method 800 includes insertion of the catheter assembly through the sacral hiatus. In an example, the posterior epidural space may be approached through a posture puncture wound in the back allowing the ligamentum flavum to be perforated. The catheter is then passed through the ligamentum flavum allowing the epidural space to be inspected and treated through the endoscope. In an exemplary embodiment, a method is provided for allowing for a catheter of a surgical apparatus to be percutaneously inserted into and to navigate the epidural space with no excision of boney structures. In some implementations, the method includes laying a patient in a prone position, identifying their sacral hiatus, determining an insertion site, and applying a local anesthetic. The insertion site can be confirmed according to several scenarios including using x-ray imaging. Next the catheter assembly of the surgical apparatus is navigated through the epidural space. In an example, the catheter assembly can be navigated through the epidural space based on pre-operative imaging (e.g., MRI imaging) target areas for fat removal. The distal end of the catheter can be used to break up adipose tissue and suction the tissue out of the epidural space.

### Locating Tissue of Interest

Locating a tissue of interest (810) to remove can be done according to several scenarios. SEL can be well demonstrated on MR imaging of the spine. In an example, locating the tissue of interest can be done by tracking claudication symptoms such as pain, burning and weakness in the legs while standing or walking. Physical exam looking for levels of spine where there is evidence of neurological dysfunction evidenced by loss of motor power or sensation. In an example, locating the tissue of interest can be done by fluoroscopic observation techniques. An example of fluoroscopic observation is described in U.S. Pat. No. 5,215,105 by Kizelshteyn, et al. entitled “Method Of Treating Epidural Lesions” the contents thereof hereby incorporated by reference. In an example, locating the tissue of interest can be done by using cameras on the endoscopic devices that can be used to give the physician or other medical personnel a detailed live view of structures within the epidural space. In an example, locating the tissue of interest can be done by using an endoscope having an elongated insertable part of the endoscope through a tube or sleeve inserted into a body vessel or cavity, or directly into the body vessel or cavity itself, such as seen in U.S. Pat. No. 5,195,541 by Obenchain entitled “Method Of Performing Laparoscopic Lumbar Discectomy”, the contents thereof hereby incorporated by reference.

In some implementations, the tissue can be visually distinguished under white light by the camera system or the user. Dura mater appears as either a blue-gray or gray-white exteriors with small blood vessels on its surface. Epidural fat appears typically yellowish in color, globular, and glistening with small blood vessel on or in it. Nerve roots are white tinged with yellow tube shape with a vessel running longitudinally down the center. Ligamentum flavum appears as white and concave tube without vessels.

According to an exemplary embodiment, locating the tissue of interest includes identifying at least one of a nerve, artery, vein, and epidural fat not to be removed within the epidural space and planning placement of an epidural surgical device based on the identification. In an example, the method includes detecting a tissue property using at least one sensor 508. In an example, the method includes navigating a distal end of the catheter based on the tissue property.

### Guiding Catheter Assembly to the Epidural Space

Guiding a catheter assembly to the epidural space (820) can be done according to several scenarios. In some implementations, a guide needle can be placed into the epidural space and a guide wire can be fed through a lumen of the guide needle. In this case, a catheter sleeve can be directed by the guide wire into the epidural space. Next the catheter assembly of the surgical apparatus can be inserted into the catheter sleeve and navigated through the epidural space. In an example, the catheter assembly can be navigated through the epidural space based on pre-operative imaging (e.g., direct visual, MM imaging) target areas for fat removal. In an aspect, navigation could be based on pre-operative imaging and target areas of epidural fat are based on MRI imaging. Specifically, pre-operative imaging can determine what level of the spine is involved and whether the excess fat in located anteriorly, posteriorly or both.

In an example, the catheter assembly can be guided within the epidural space using ultrasound guided techniques. In an example, the catheter assembly can be guided within the epidural space using guidance positioning system such as SonixGPS™ from Sonix Medical Corporation (Peabody, Ma.). In an example, the catheter assembly can be guided within the epidural space using real-time three-dimensional or four-dimensional ultrasonography. In an example, the catheter assembly can be guided within the epidural space using ultrasound imaging with pre-acquired three-dimensional images of the spine. In an example, the catheter assembly can be guided within the epidural space using ultrasound through needle. In an example, the catheter assembly can be guided within the epidural space using needle through ultrasound. In an example, the catheter assembly can be guided within the epidural space using machine vision. In an example, the catheter assembly can be guided within the epidural space using acoustic radiation force impulse imaging. In an example, the catheter assembly can be guided within the epidural space using fluoroscopy. Confirming catheter assembly entry proximal to the tissue of interest within the epidural space

The catheter assembly entry can be confirmed proximal to the tissue of interest within the epidural space (830) according to several scenarios. In an example, the catheter assembly entry can be confirmed proximal to the tissue of interest within the epidural space using loss of resistance technique. In an example, the catheter assembly entry can be confirmed proximal to the tissue of interest within the epidural space using membrane in syringe technique. In an example, the catheter assembly entry can be confirmed proximal to the tissue of interest within the epidural space using an epidural balloon. In an example, the catheter assembly entry can be confirmed proximal to the tissue of interest within the epidural space using EpiDrum® from Exmoor Innovations (Taunton, UK). In an example, the catheter assembly entry can be confirmed proximal to the tissue of interest within the epidural space using the Episure™ AutoDetect™ from Indigo Orb, Inc. (Orange County, Calif.). In an example, the catheter assembly entry can be confirmed proximal to the tissue of interest within the epidural space using an auditory and visual display of a pressure wave. In an example, the catheter assembly entry can be confirmed proximal to the tissue of interest within the epidural space using bioimpedance using the one or more sensors 508. In an example, the catheter assembly entry can be confirmed proximal to the tissue of interest within the epidural space using optical sensing including optical reflectance spectroscopy and optical coherence tomography. In an example, the catheter assembly entry can be confirmed proximal to the tissue of interest within the epidural space using at least one of an epidural stimulation test, an electrocardiography guided system, epidurography, epidural pressure waveform analysis, near-infrared tracking system, ultrasound. Controlled suctioning of the tissue of interest

According to an exemplary embodiment, the method includes controlled suctioning of the tissue of interest (840). Suctioning typically calls for placing the catheter tip in close proximity to the substance targeted for removal. The negative pressure will jettison the material, liquid or solid without further action by the operator. However, excision of the semi-solid fat is aided by repeated piston-like action to rip or tear the adipose tissue. Motionless catheter, which works well for sucking liquid, is not well suited for removal of lipids. Active motion by the operator breaks up the adipose tissue into small morsels which can be effectively suctioned. Moving catheter assembly 700 (FIG. 7) can be well suited for providing the piston-like action to rip or tear the adipose tissue.

In some implementations, the controlled suctioning is a selective suctioning of tissue within the aqueous environment. Removing bodily fluid can be dangerous, therefore, the suctioning can monitor an amount of fluid-to-tissue volume. In some implementations, the controlled suctioning is a pulsed suction. In an example, the pulsed suction can suction tissue, applying a stress on the tissue for a certain duration, and then pause to allow for the tissue to relax. In some implementations, the one or more sensors is configured to sense a property of the targeted tissue between each suction. In an aspect, tissue sensing is configured to allow for confirmation of tissue type before each suction removal. In some implementations, the controlled suctioning of the tissue of interest will only start when electrical contacts sense tissue within the lumen of the catheter.

### Pressurizing a Bleeding Site

In a case of leakage (e.g., hemorrhage) in the surgical site, the surgical apparatus can be configured to suction fluid such as CSF or blood and control the leakage by pressurizing a leaking site and providing a view enabling a hemostatic site to be treated.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. A surgical system for removing adipose buildup from epidural space of a patient spine comprising:
a surgical apparatus including a handle portion and a catheter assembly having a steerable distal tip configured for navigating epidural space of the spine;
an endoscopic camera system configured to capture images from the epidural space and to identify a tissue of interest; and
a vacuum source in communication with the catheter assembly of the surgical apparatus and configured to provide a negative pressure through a lumen of the catheter assembly to the tissue of interest;
a discharge tank configured to capture suctioned tissue from the catheter assembly; and
wherein the surgical apparatus further includes at least one fiber optic cable in communication with the endoscopic camera system.

2. The surgical system of claim 1, wherein the vacuum source comprises a regulator configured to automatically stop based on a detected tissue property.

3. The surgical system of claim 1, further comprising a morcellator configured to break up the suctioned tissue of interest.

4. The surgical system of claim 1, further comprising an agent source in communication with a lumen of the catheter assembly; and wherein the agent source is configured to condition the tissue of interest.

5. A surgical apparatus for removing tissue from epidural space of the patient spine, the surgical apparatus comprising:
a handle portion a catheter assembly having a steerable distal tip configured for navigating epidural space of the spine;
at least one fiber optic cable in communication with an endoscopic camera system configured to allow visualization of the interior of the epidural space and to identify a tissue of interest; and
a lumen of the catheter assembly in communication with a vacuum source configured to provide suction to the tissue of interest.

6. The surgical apparatus of claim 5, wherein the distal tip of the catheter assembly further includes an adjustable suction tip configured to modify a suction area.

7. The surgical apparatus of claim 5, further comprising a manipulator configured to manipulate a portion of tissue obstructing the tissue of interest.

8. The surgical apparatus of claim 5, further comprising: an actuator configured to vibrate the catheter assembly.

9. The surgical apparatus of claim 5, wherein the distal tip of the catheter assembly has a blunt shape configured to prevent piercing of the thecal sac.

10. The surgical apparatus of claim 5, further comprising a marker for confirming catheter assembly within in the epidural space for imaging guidance.

11. The surgical apparatus of claim 5, further comprising one or more sensors configured to sense a tissue property.

12. The surgical apparatus of claim 11, wherein the one or more sensors is configured to sample tissue for detection.

13. The surgical apparatus of claim 11, wherein the one or more sensors is configured for performing electrical recordings.

14. The surgical apparatus of claim, wherein the one or more sensors includes one or more pressure sensors configured to detect a mechano-tissue response and to identify a tissue property.

15. The surgical apparatus of claim 11, wherein the distal tip of the catheter assembly further includes at least two suction tips configured to modify a suction area; and
wherein each suction tip of the at least two suction tips includes at least one electrode configured to interact with another electrode on another suction tip.

16. A method for removing adipose buildup from epidural space of a patient, the method comprising:
locating a tissue of interest within the epidural space;
guiding a catheter assembly for a surgical apparatus within the epidural space;
confirming the catheter assembly entry proximal to the tissue of interest into the epidural space; and
controlled suctioning of the tissue of interest.

17. The method of claim 16, further comprising at least one of an ultrasound-assisted lipectomy and radiofrequency-assisted lipectomy.

18. The method of claim 16, further comprising conditioning the tissue of interest.

19. The method of claim 16, wherein the controlled suctioning is a pulsed suction.

20. The method of claim 16, further comprising sensing a property of the tissue of interest between each suction iteration.
